(19) United States
(12) Patent Application Publication
(10) Pub. No.: US 2010/0286714 A1
(43) Pub. Date: Nov. 11, 2010

(54) INSERTER DEVICE WITH CONTROLLED ACCELERATION

(76) Inventors: Steffen Gyrn, Ringsted (DK); Henrik Tipsmark, Roskilde (DK)

Correspondence Address: BRINKS HOFER GILSON & LIONE, P.O. BOX 10395, CHICAGO, IL, 60610, US

(21) Appl. No.: 12/669,000

(22) PCT Filed: Jul. 3, 2008
(86) PCT No.: PCT/EP2008/058597

§ 371 Date: May 20, 2010

Related U.S. Application Data
(60) Provisional application No. 60/950,496, filed on Jul. 18, 2007.

(30) Foreign Application Priority Data
Jul. 18, 2007 (DK) PA 2007 01062

(51) Int. Cl. A61B 17/10

(52) U.S. Cl. 606/139

(57) ABSTRACT

The invention concerns an inserter device for inserting a medical device into the subcutaneous or intramuscular area of a patient. More specifically, this invention relates to an inserter device comprising means for providing a controlled and defined acceleration and deceleration of a penetrating member. The inserter device (200, 500) according to the invention comprises a housing (201, 221, 251; 501, 502, 503) encompassing said penetrating member (105, 243), a rotating member (204, 300, 400, 512) and driving means (203, 561) for rotating the rotating member (204, 300, 400, 512) around a rotating axis. The rotating member (204, 300, 400, 512) comprises transformation means (216, 246, 521) transforming the rotational movement into a longitudinal movement of the penetrating member (105, 243) in the direction of insertion and the transformation means (226, 246, 521) comprises controlling means providing a controlled variation of the velocity of the penetrating member (105, 243) in the direction of insertion.

### TECHNICAL FIELD OF THE INVENTION

The invention concerns an inserter device for inserting a medical device into the subcutaneous or intramuscular area of a patient. More specifically, this invention relates to an inserter device comprising means for providing a controlled and defined acceleration and deceleration of a penetrating member.

### BACKGROUND OF THE INVENTION

Inserter devices, also called injectors, are commonly used in the medical field for inserting medical devices such as infusion sets and the like, in a semi-automated fashion through the skin of a patient.

It is known that most patients, especially children, are afraid of sharp objects, such as injection needles and other penetrating devices, commonly used for medical treatment and therapy. This fear is often irrational, and it may hamper an appropriate medical treatment.

A further issue related to insertion of medical devices is the risk of contamination of the penetrating member before or during application. This can easily lead to the introduction of an infection to a patient, e.g. through a contaminated insertion needle. The longer such a needle is exposed, the higher the risk of accidental contamination, e.g. by touching the needle with a finger, bringing the needle in contact with an unclean surface, or by airborne contamination, aerosol contamination and the like. Depending on the nature of the contamination (e.g. comprising virus, bacteria, fungus, yeast and/or prion) combined with the general health status of the patient, the resulting infection can rapidly turn into a life threatening situation.

It is well known that contact with an infected, used needle especially in hospital environments can be life-threatening, and the risk of accidental exposure to contaminated material must be minimized.

Inserter devices and the like are known in the art. EP 1 011 785 relates to an injector for a subcutaneous infusion set, EP 1 044 028 concerns an inserter device for an insertion set.

Commonly, insertion of e.g. a cannula or injection needle provides discomfort and pain. An objective of the present invention is to reduce discomfort and pain to a minimum.

Penetration of the skin with a medical device results in tissue damage. It is an object of the invention to reduce tissue damage.

Medical devices can be sensitive; it is thus an object of the invention to provide an inserter device that protects sensitive medical devices before and during insertion into a patient.

Known devices do not provide a defined and controlled insertion and retraction speed, combined with defined and controlled acceleration and deceleration of the penetrating member to be inserted into a patient.

As none of the known devices solve the problems and issues listed above, there is an obvious need in the art for an insertion device, which addresses the issues discussed above, and which provides a controlled insertion of a penetrating member through defined acceleration and deceleration.

### SUMMARY OF THE INVENTION

The current invention provides an insertion device, where a controlled acceleration and deceleration of a penetrating member is provided. In the present application a penetrating member is defined as a part that passes through or penetrates the skin surface of a patient, i.e. the penetrating member can be an insertion needle combined with a soft cannula, a hard self-penetrating cannula, a self-penetrating sensor or a sensor combined with an insertion needle.

A first aspect of the invention concerns an inserter device, where a rotational movement is transformed into a longitudinal insertion movement, wherein the axis of said rotational movement and the axis of said longitudinal insertion movement are essentially parallel and/or aligned with each other.

A second aspect of the invention relates to an inserter device, where a rotational movement is transformed into a longitudinal insertion movement, wherein the axis of said rotational movement and the axis of said longitudinal insertion movement are essentially perpendicular to each other.

Thus, the present invention provides an inserter device as defined by claim 1. This inserter device comprises a housing encompassing a penetration member, and the inserter device comprises means for providing a defined acceleration and deceleration of said penetration member. The defined acceleration and deceleration is achieved as a result of applying an essentially constant force and not by varying the applied force.

According to one embodiment the inserter device comprises a housing encompassing said penetrating member, a rotating member and driving means for rotating the rotating member around a rotating axis, the rotating member comprises transformation means which means transforms the rotational movement into a longitudinal movement of the penetrating member in the direction of insertion wherein the transformation means comprises controlling means providing a controlled variation of the velocity of the penetrating member in the direction of insertion.

According to one embodiment the rotating member's rotation axis is parallel to the direction of insertion of the penetrating member. In one case the rotating member's rotation axis is aligned with the axis of insertion of the penetrating member.

According to one embodiment the transformation means comprises a groove on a surface of a body part of the rotating member corresponding to a protruding part connected to the penetrating member. According to this embodiment the controlling means can comprise the slope of the groove as the groove extent in a direction which is not parallel to the direction of insertion, The transformation could comprise any corresponding parts on respectively the rotating member and the penetrating member which corresponding parts can provide a slidable fit.). The groove can be continuous and the slope of the groove can be defined in a system of coordinates having an ordinate axis parallel to the rotation axis of the rotation member. At least a part of the groove can have a negative slope or a constant negative slope in the whole length of the groove when providing a movement where the longitudinal moving member is moving towards the skin of the patient. The negative slope of the groove can be decreasing as the longitudinal moving member moves toward the skin of the patient. The groove can be continuous and at least a part of the groove can have a positive slope or the groove can have a constant positive slope in the whole length of the groove in a system of coordinates having an ordinate axis parallel to the rotation axis of the rotation member, when providing a movement where the longitudinal moving member is moving away from the skin of the patient. The positive slope of the groove can be decreasing as the longitudinal moving member moves away from the skin of the patient.

According to one embodiment the body part of the rotating member is cylindrical and the groove is formed in the outer surface of the body part, means corresponding to the groove are formed as at least one inward protruding part on an inner surface of the longitudinal moving member.

According to one embodiment the rotating member's rotation axis is not parallel to the direction of insertion of the penetrating member, e.g. the rotating member's rotation axis can be orthogonal to the direction of insertion of the penetrating member. If the angle of the rotation axis deviates a few degrees from orthogonal it will still be considered “orthogonal” according to this invention. Also the rotating member can be a shaft, which shaft can be provided with one or more discs protruding in relation to the shaft.

Such a shaft can be a crank shaft provided with two discs that are attached orthogonally and concentrically onto said crank shaft, so that crank shaft and discs share the same rotation axis.

According to one embodiment the direction of insertion of the penetrating member is either essentially perpendicular to the patient's skin surface, i.e. insertion is provided at an insertion angle αᵢₙₛ around 90° where the surface of the patients skin is considered to constitute the base line of 0°, or 0°<αᵢₙₛ≦20°, or 20°<αᵢₙₛ≦40°, or 40°<αᵢₙₛ≦60°, or 60°<αᵢₙₛ≦80°.

According to one embodiment the central axis of the inserter device is essentially perpendicular to the patient's skin surface when the inserter device is placed in a position ready for insertion, i.e. the inserter device has a central axis angle α_{centre} around 90° where the surface of the patients skin is considered to constitute the base line of 0°, or 0°<α_{centre}≦20°, or 20°<α_{centre}≦40°, or 40°<α_{centre}≦60°, or 60°<α_{centre}≦80°.

According to one embodiment the direction of insertion of the penetrating member is parallel to the central axis of the inserter device, i.e. has a deflection angle α_{deflection}=0° from the central axis, or 0°<α_{deflection}<90°, or 10°<α_{deflection}<80°, or 30°<α_{deflection}<60°.

According to one embodiment the transformation means transform a rotation of the rotating member of more than approximately 180°, where approximately means±10°, into a longitudinal movement, said longitudinal movement providing an insertion of the penetrating member. Especially the transformation means can transform a rotation of more than 360°, or more than 1.5 revolutions, or more than 2 revolutions, of the rotating member into a longitudinal movement, said longitudinal movement providing an insertion of said penetrating member.

According to one embodiment the penetrating member comprises a soft cannula and an introducer needle. E.g. the introducer needle can be part of the inserter device, and the introducer needle can then be removed from a medical device comprising a soft cannula after insertion of the penetrating member.

According to this embodiment continued rotation of the rotating member in the same direction of rotation can provide insertion of the penetrating member followed by retraction of the introducer needle or rotation of the rotating member in the opposite direction of rotation after insertion of the penetrating member can provide retraction of the introducer needle.

E.g. a rotation of the rotating member of approximately 180°, where approximately means±10°, can provide retraction of said introducer needle or a rotation of the rotating member of less than 180°, or less than 150°, or less than 120°, or less than 90°, or less than 60°, or less than 30° provides retraction of the introducer needle.

### BRIEF DESCRIPTION OF THE DRAWINGS

A detailed description of embodiments of the current invention will be made with reference to the accompanying figures, wherein like numerals may designate corresponding parts in different figures.

FIG. 1: A medical device comprising a penetrating member, a body part and a mounting pad.

FIG. 2: Embodiment of an inserter device and its main components.

FIG. 3: Alternative embodiment of an inserter device and its main components.

FIG. 4: An embodiment of an inserter device with activation means, including close-up of top section.

FIG. 5: Embodiment of a rotating member and a piston with introducer needle.

FIG. 6: Alternative embodiments of rotating means.

FIG. 7: Detailed view of an embodiment of a piston with transformation means (protrusion).

FIG. 8: Cross section of an embodiment of an inserter device with medical device before insertion.

FIG. 9: Cross sections of an embodiment of an inserter device with medical device at positions before insertion (A), inserted (B) and retracted (C).

FIG. 10: Different views of embodiments of an inserter device and a medical device.

FIG. 11: Alternative embodiments of transformation means comprising spiral spring, rotating means and piston.

FIG. 12: Embodiment of an inserter device with activation means.

FIG. 13: Illustration of the mode of action of an embodiment of an inserter device with transformation means.

FIG. 14: Schematic representation of embodiments of inserter devices with external activation means.

FIG. 15: Two principles of providing controlled variations of insertion speed and acceleration/deceleration as a function of angular velocity.

FIG. 16: Semi-transparent view of an embodiment of an insertion device with crankshaft

FIG. 17: An embodiment of an insertion device with crankshaft with partial cross section through the lower section including medical device with penetrating member.

FIG. 18: An embodiment of an insertion device with crankshaft with partial cross section through the top section.

FIG. 19: Illustration of the mode of action of an insertion device with crank-shaft.

### DETAILED DESCRIPTION OF THE INVENTION

According to the invention, different medical devices can be inserted into the subcutaneous or intramuscular region of a patient. Such medical devices may comprise e.g. infusion sets or the infusion part of an infusion set, sensor devices comprising one or more inserted sensors, port devices which only comprise a body with a restricted access for replacing repeated injections with syringes, or any other device having a penetrating member inserted into the subcutaneous area or intramuscular area of a patient.

It is one of the objects of the present invention to provide an inserter device that allows for a controlled, defined and adjustable insertion of a medical device into a patient. Such a controlled, defined and adjustable insertion can be achieved by controlling the speed of insertion, and optionally, also the speed of retraction of parts of the inserter device, such as insertion needles commonly used to insert a medical device with for example a soft cannula, which cannot be inserted directly. However, the inventors not only provide an inserter device with a controlled speed of insertion, but a novel and inventive inserter device, where speed as well as acceleration and deceleration of insertion can be controlled. Thereby, significantly improved reliability, ease of operation, and user friendliness are provided due to controlled insertion characteristics and features.

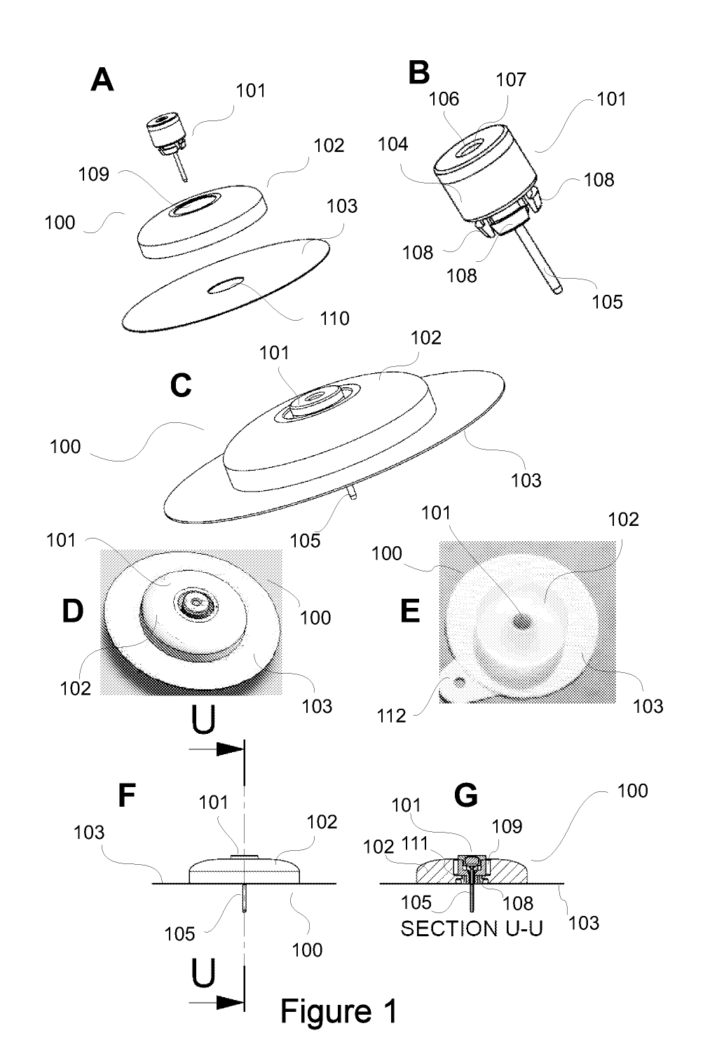

FIG. 1 shows different embodiments of a medical device 100 that can be inserted according to the invention. The medical device shown in FIG. 1A comprises a cannula holding part 101, a body 102 and a mounting pad 103, as depicted in FIG. 1A.

FIG. 1B shows an embodiment of a cannula holding part 101 comprising a top section 104 and a cannula 105. The top section 104 comprises an opening 106, closed by a sealing device 107. Towards the bottom of top section 104, locking means 108 are provided. An internal chamber (not shown) is defined within top section 104; said internal chamber is in connection with cannula 105.

As seen in FIG. 1A, the body 102 of the medical device 100 comprises an opening 109 encompassing at least a part of the cannula holding part 101. Likewise, the mounting pad 103 comprises an opening 110, which is at least as wide as or wider than the cannula 105 or cannula holding part 101. In another embodiment, one or more penetrating member(s), such as an injection needle, inserter needle or cannula, are injected across the mounting pad 103, said mounting pad having no opening 110.

Often, a mounting pad 103 is used to ensure the appropriate contact of the medical device 100 with the skin of the patient. This mounting pad 103 may be attached to the underside of the body 102 of the medical device 100. Alternatively, the mounting pad 103 is attached to the skin of the patient, and the medical device 100 is inserted through the mounting pad 103 or through an opening 110 in the mounting pad 103.

FIG. 1C illustrates another view of the medical device 100 shown in FIGS. 1A and B. FIGS. 1 D and E illustrate other embodiments of a medical device 100. As seen in FIG. 1 E, the mounting pad comprises a release liner comprising a flap 112, in order to remove the release liner before application of the medical device.

FIGS. 1 F and G show a side view and a cross section of the medical device 100. In Figure G, the interlocking means 111, which provide a connection between the locking means 108 and the body 102, can be seen.

### Rotation Axis and Insertion Axis are Parallel

In a first aspect, the invention concerns an inserter device, where a rotational movement is transformed into a longitudinal insertion movement, wherein the axis of said rotational movement and the axis of said longitudinal insertion movement are essentially parallel to each other. The following section describes such embodiments, where said axis of said rotational movement and the axis of insertion are essentially in alignment.

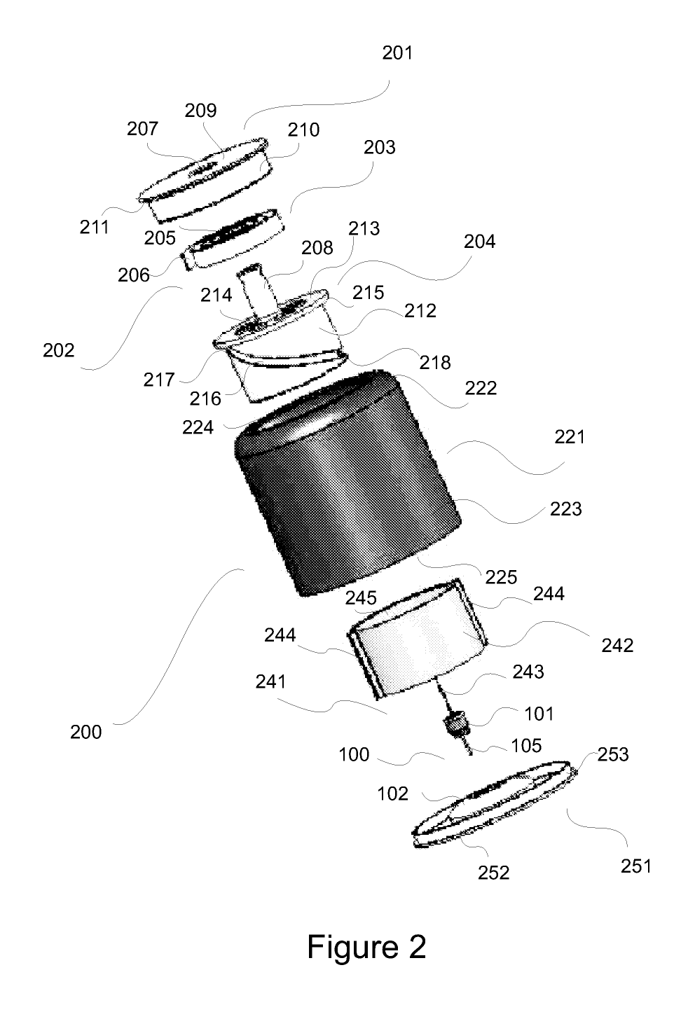

FIG. 2 shows an exploded view of the main components of an inserter device 200 according to one embodiment of the invention. The inserter comprises (i) a top section 201; (ii) rotating means 202 comprising a spiral spring 203 and a rotating member 204; (iii) a middle section 221; (iv) longitudinally moving means 241; and (v) a bottom section 251. Said sections 201, 221 and 251 define essentially the outer dimensions of the inserter device 200, as well as an inner cavity (not shown). Within said inner cavity the rotating means 202 as well as the longitudinally moving means 241 are provided.

FIG. 2 reveals that the different sections and means are predominantly aligned and oriented in or around the central axis of the inserter device, and the axis of insertion is essentially the same as the central axis of the inserter device.

The following section provides a detailed, top to bottom description of the components illustrated in FIG. 2.

Ad (i) The top section 201 comprises a top part 209 and a body part 210. At the centre of the top part 209 attachment means 207 are provided for attachment of part (shaft 208, see below) of the rotating member 204. Such attachment means 207 can comprise an opening or a bearing. The top part 209 is essentially flat and formed like a disk. The diameter of the top part 209 exceeds the diameter of the body part 210, thus forming a protrusion 211. The body part 210 is essentially shaped like a hollow cylinder, and the inner diameter of body part 210 exceeds the outer diameter of the spiral spring 203 upon assembly of the inserter device 200. One or more attachment means (not shown) for the spiral spring 203, such as for the outer section 206, can be provided within top part 209 and/or body part 210 of the top section 201.

Ad (ii) the rotating means 202, comprising a spiral spring 203 and a rotating member 204. Said spiral spring 203 comprises an inner end 205 positioned towards the centre of said spring 203, and an outer end 206 positioned at the periphery. Both inner 205 and outer end 206 can be shaped individually and independently, for example forming a flap, such as by bending the spiral member in an inwards or outward fashion. As depicted in this embodiment, the outer end 206 is formed by bending a section of the spiral member outwards. Likewise, the inner end 205 of the spiral spring 203 can also be shaped in a corresponding fashion. The spiral spring 203 rests between the top section 201 and the rotating member 204. The outer diameter of the spiral spring 203 is of similar size or smaller than the inner cavity of top section 201, at least during assembly of the inserter device 200.

The rotating member 204 comprises a body part 212, a top part 213, and a shaft 208. The top part 213 is essentially flat and formed like a disk, and it can comprise one or more openings 214. The diameter of the top part 213 exceeds the diameter of the body part 212, thus forming a protrusion 215. The body part 212 is of cylindrical shape, and it can comprise one or more openings (not shown), said openings being in connection with the one or more opening 214, thus forming one or more channels from the top part 213 to the bottom end (not shown) of the body part 212. One or more grooves 216 formed in the outer surface of the body part 212 extend from the upper end 217, i.e. the distal end which is farthest away from the patient during insertion, to the lower end 218, i.e. the proximal end which is closest to the patient during insertion, of the rotating member 204. Furthermore, the rotating member 204 comprises a shaft 208, said shaft 208 protruding upwards, and being aligned with the rotating axis of the rotating member 204. The length of the shaft 208 exceeds the height of the spiral spring 203. In order to accommodate the spiral spring 203, the diameter and shape of the shaft 208, as well as the inner section of the spiral spring 203 are dimensioned in such a way that—upon assembly of the inserter device 200—a major part of the spiral spring 203—such as the whole spiral spring 203 minus a part of the inner section 205—surrounds the shaft 208, upon assembly of the inserter device 200.

Ad (iii) the middle section 221 is the largest component of the inserter device 200, defining essentially the outside dimensions of the inserter device 200. The middle section 221 is essentially of hollow-cylindrical shape, and can comprise a top part 222 and a body part 223. Top part 222 and body part 223 can be to different units that are joined together, e.g. by melding or gluing, or they are part of the same (work) piece. Both top part 222 and body part comprise openings at both end, i.e. top opening 224 and bottom opening 225, respectively. Middle section 221 thus defines the central cavity of the insertion device, which is wide enough to encompass a major part of the above-mentioned rotating means 202, as well as the longitudinally moving means 241, and the medical device 100 to be inserted. The top part 222 can be rounded, as depicted in FIG. 2, and can comprise a recess (not shown) in order to encompass at least a part of the protrusion 211 of the top section 201. Furthermore, body part 223 and optionally top part 222 comprise inner guiding means (not shown), such as one or more slots (not shown), in order to govern the longitudinal movement of the longitudinal moving means 241. The outer shape of the inserter device 200 is predominantly defined by the shape of the middle section 221, which can be round, elliptical, square, symmetric across one line, rotational symmetric, or even asymmetric, e.g. in order to provide a better grip for left-handed versus right-handed persons, as well as for people with small hands compared to people with larger hands, or prostheses. Thereto, special grip-means (not shown) can be provided.

Ad (iv) The longitudinal moving means 241 comprise a piston 242, an insertion needle 243, longitudinal guiding means 244, and an inner cavity 245, which extends from the top of the piston downwards. Said piston 242 is of essentially hollow-cylindrical shape. The inner diameter of the piston 242 is smaller than the outer diameter of the body part 212 of the rotating member 204, but it can be smaller than the diameter of the top part 209 of the rotating member 204. The height of the piston 242 can be essentially the same, smaller or larger than the height of the body part 212 of the rotating member 204. At least a major portion of the rotating member 204, such as a major part of the body part 212 fit into the inner cavity 245. One or more transformation means (not shown) can be provided, which are attached and protruding from the inner wall of the piston 242, said transformation means fit into the groove 216, thus transforming a rotational movement of the rotating member 204 into a longitudinal movement of the piston 242. At the bottom of the piston 243, such as at the centre or off centre of said bottom, an insertion needle 243 is attached via attachment means (not shown). The insertion needle extends perpendicular to the bottom of the piston 243, and is aligned in the orientation of insertion. In the depicted embodiment in FIG. 2, the insertion needle 243 is at least partially inserted into the cannula holding part 101, comprising a body part 101 and a soft cannula 105. Thus, cannula holding part 101 and piston 242 are connected via the insertion needle 243.

Ad (v) The bottom section 251 of the inserter device 200 comprises a bottom part 252 and a ring-shaped part 253. Bottom part is essentially shaped like a disk, and can have a larger diameter than the ring shaped part 253. In the depicted embodiment (FIG. 2), the bottom part 252 can actually be provided by the mounting pad 103 of the medical device 100 (see FIG. 1 for details). Thus, the bottom part 252 may comprise a disposable liner and a flap 105. As depicted and if appropriate, the body 102 of a medical device 100 rests on the bottom part 252, and is held in an suitable position, such as with the central cavity of the body 102 of the medical device being aligned with the cannula holding part 101 and the insertion needle 243. The outer diameter of the ring-shaped part 253 is the same or smaller than the inner diameter of the bottom opening 225 of the middle section 221, and the ring-shaped part 253 fits into the body part 223 of the middle section 221. In an alternative embodiment of the invention, the ring-shaped part 253 is omitted, and the bottom opening 225 of the middle section is sealed by the mounting pad 103 of the medical device 100 to be inserted.

Generally, the mounting pad's adhesive strength is sufficiently strong to ensure that the medical device remains on the skin of the patient after insertion, and only the insertion needle 243 is removed through the cannula 105, while the remaining parts of the medical device 100 remain in place. In an alternative embodiment of the current invention, the medical device 100 is inserted through a further medical device.

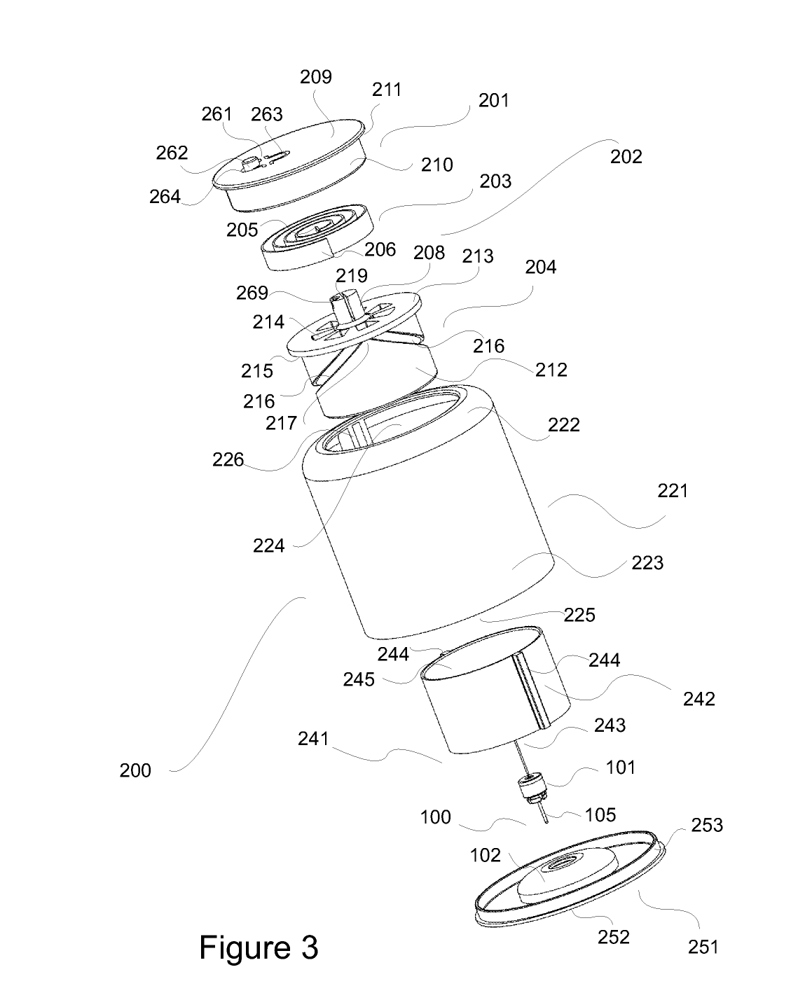

FIG. 3 shows an exploding view of an alternative embodiment of the current invention. Please refer to FIG. 2 for explanation and numbering of the relevant components and features are depicted in FIG. 3 as in FIG. 2.

Several additional and/or different features become apparent, when rotating the inserter device 200 by approximately 90° in anti-clockwise direction:

(i) The inserter device 200 comprises activation means 261, situated off-center of the top part 209 of the section 201. The activation means comprise a button 262, one or more apertures 263, 264 across the top part 209, and a notch 269, situated towards the upper end of the shaft 208, slightly off-center of the rotating element 204.
(ii) An embodiment of attachment means 219 for the inner end 205 of the for spiral spring 203 on the shaft 208 of the rotating member 204 are shown.
(iii) An embodiment of body part 212 of the rotating member 204 is shown, revealing an embodiment of the groove 216, showing essentially the first and last quarters oft the groove 216. It is seen that the groove 216 is not continuous.
(iv) An embodiment of inner guiding means 226, residing within the central cavity of the middle section 221 is shown. In this embodiment, the guiding means form a longitudinal groove, extending essentially from the top part 222 to body part 223 (not shown). The height of the guiding means 226 equals approximately half the difference between the diameter of the bottom opening 225 minus diameter of top opening 224.

Ad (i): According to one embodiment of the invention, the inserter device 200 is activated by actuated the button 262 by pushing and/or sliding and/or rotating and/or pivoting from a position 1, where the rotating member 204 is impeded from rotating around its rotating axis, to a position 2, where the rotating member 204 can rotate around its rotating axis. According to the depicted embodiment, the button 262—which has a rod-like shape—fits into the notch 269 of the shaft 208 in position 1, thereby impeding the rotating member 204 from rotating. Upon actuation of the button 262—such as sliding or pushing the button 262 outwards, i.e. from a position, where the button 262 resides essentially within aperture 263 towards a position, where the button 262 resides essentially within aperture 264—the spiral spring 203, which is in an activated state, is allowed to reach a less activated, more relaxed state, whereupon a rotating movement of the rotating member 204 is provided.

In an alternative embodiment, the button 262 is lifted upwards upon actuation, thereby leaving notch 269, thereby allowing for rotation of the rotating member 204.

In order to provide energy for rotating the rotating member 204, the spiral spring 203 has to be converted from an essentially relaxed state to an activated state.

This activated or loaded state can be provided by preventing either the inner end 205 or the outer end 206 from moving, and rotating either inner end 205 or outer end 206, either in a direction, where the spiral spring 203 becomes more closely packed towards the center 205 of the spring, or where the spiral spring 203 becomes more closely packed towards its outer end 206. Relaxation of the spiral spring 203 occurs in the opposite direction of rotation than its activation.

In the depicted embodiment in FIG. 3, the inner end 205 of the spiral spring 203 is situated within a groove 219 of the shaft 208. The length of the groove 219 is essentially the same or shorter than the length of the shaft 208. The width of the groove 219 is essentially the same or wider than the width of the leaf of the spiral spring 203. Upon relaxation of the spiral spring, the inner end 205 rotates, and relays this rotating movement to the shaft 208 and thus to the rotating member 204.

Attachment means for the outer end 206 of the spiral spring, residing within the upper section 201 are not shown.

According to one embodiment of the invention, the inserter device 200 is provided in a loaded state to the user.

In a further embodiment, securing means are provided in order to prevent unintentional activation of the inserter device. Such securing means can comprise mechanical, electromechanical or electronic means, or a combination of mechanical, electromechanical or electronic means.

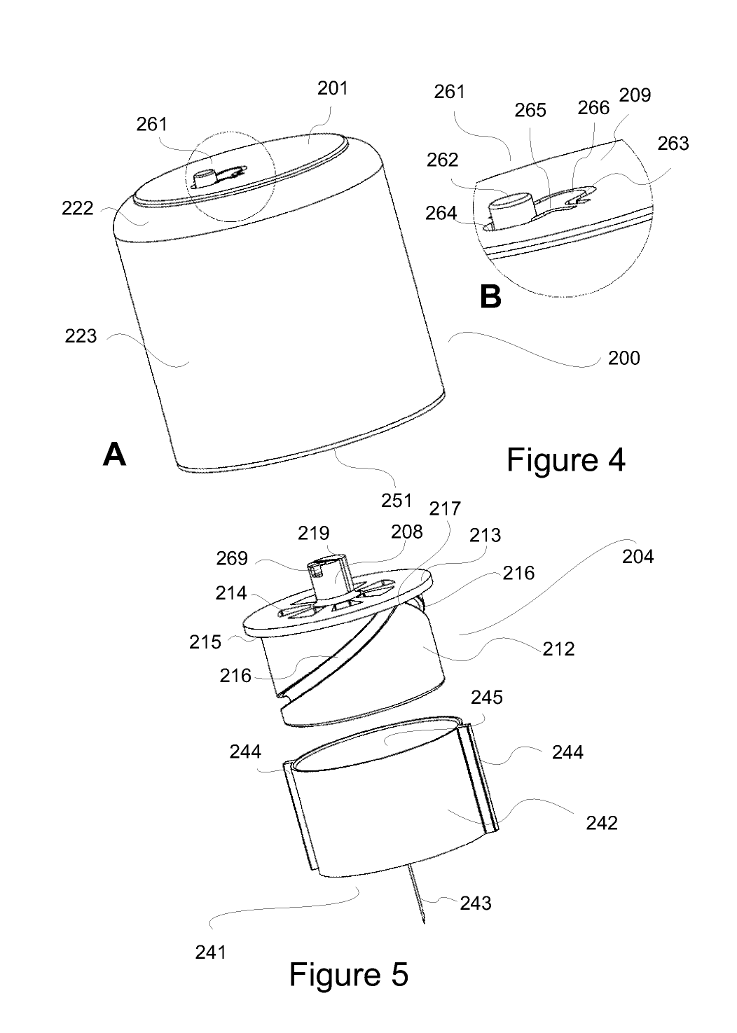

FIG. 4 shows an embodiment of an assembled inserter device 200 according to the current invention, with activation means 261 situated off center on the top section 201 (FIG. 4 A). Top section 201, middle section and bottom section 251 are indicated. A close-up of the activation means 261, comprising a button 262, and a cavity 265 and optional retention means 266 are shown in FIG. 4B. The cavity 265 extends radially, starting from a near-centre position 263, to a position 264 further away from the centre, but still well within the top section 201. The cavity 265 is rounded at inner most position 263 and outer most position 264, with a radius exceeding the radius of the button 262. The inserter device 200 can be activated by manipulating button 262 as described above. In one embodiment of the invention, the insertion device is activated by bringing button 262 from a position at or near the near-centre position 263 to a position 264 further away from the centre of the top section 201. The button 262 extends across top section 201 and fits into the notch 269 of the shaft 208 of the rotating member 204, for example in an activated position. Retention means 266 provides a resistance against unintentional activation of the device. In one embodiment of the invention, retention means 266 comprise an elastic member, such as a spring.

In an alternative embodiment, activation of the inserter device 200 is achieved by activating button 262, which activates a rocking mechanism (not shown), which comprises a blocking member (not shown) that is removed from a position within notch 269 of the activated or loaded rotating member 204. Thereby, the rotating member is no longer restricted from rotating.

FIG. 5 shows a detailed view of rotation member 204 and a piston 241 according to an embodiment of the invention, such as the one illustrated in FIG. 3. Compared to FIG. 3, rotation member 204 and a piston 241 are rotated approximately 45° in anti-clockwise direction. In this view, the upper end 217 of the rotating member 204 is seen; this is where groove 216 starts and ends. In one embodiment, the groove is not continuous. In another embodiment, the groove is continuous, i.e. without a start and/or endpoint.

In the current embodiment, approximately half a rotation of the rotating member 204, i.e. approximately 180°, are converted into a longitudinal movement of the piston 241, where the length of said longitudinal movement is essentially defined by the lead, i.e. the distance parallel to the axis between the start position of the groove 216 at the upper end 217 and at the lower end 218. At the bottom of the piston 242, an insertion needle 243 is attached.

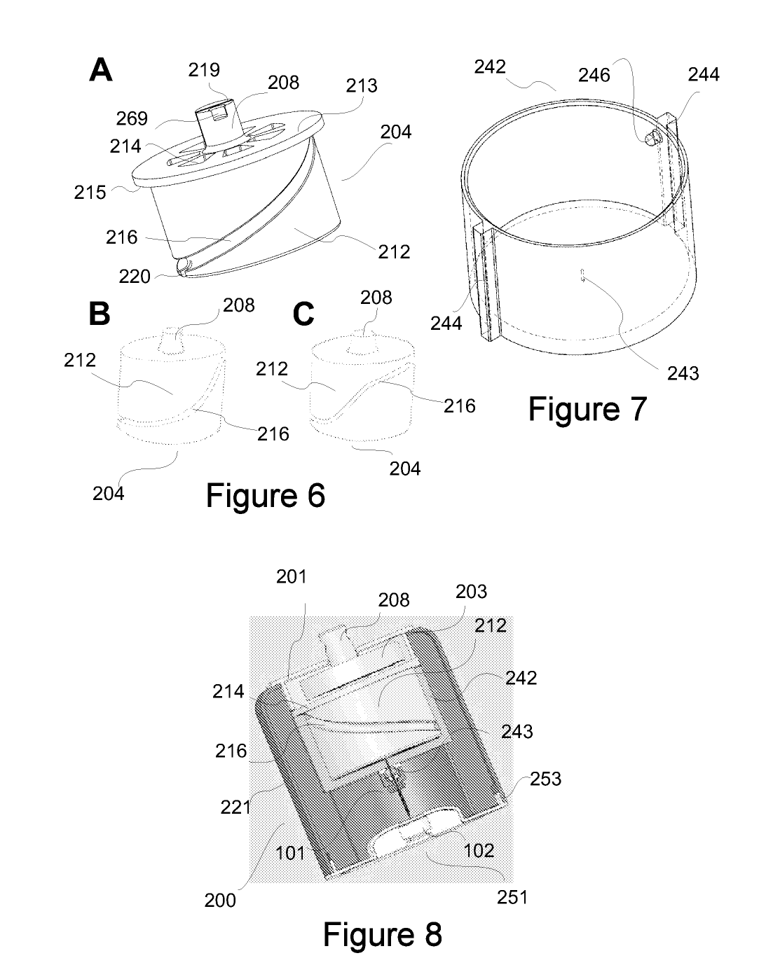

FIG. 6 shows different embodiments of rotating members 204 according to the invention. FIG. 6 A presents a further view of the rotating member 204, similar to FIGS. 3 and 5, but with an additional rotation of approximately 45° compared to FIG. 5. A notch 220 is seen at the lower end of rotating member 204, where the groove 206 reaches the lowest position. In one embodiment of the invention, notch 220 has no practical function during application of the device, but has a function during assembly and/or manufacturing of the inserter device 200, where it facilitates assembly or makes assembly possible. In another embodiment, notch 220 is not present.

FIGS. 6B and 6C show alternative embodiments of the rotating member 204. In these embodiments, the rotating member 204 does not comprise a top part 213, and no protrusion 215. The shaft 219 can vary in form and shape, and in the depicted embodiments, the shaft is of essentially round diameter, though wider at the bottom than at the top, i.e. wider towards the body part 212 of the rotational member 204. The top surface of the shaft 219 is essentially flat. In another embodiment of the invention, the top surface of the shaft 219 is concave. In a further embodiment of the invention, the top surface of the shaft 219 is convex.

In the embodiments depicted in FIG. 6 A, B and C, different positions of the respective grooves 216 on the rotating member 204 are provided. An inserter device according to the present invention will reveal different speeds and directions of the longitudinal movement of the piston 241 during one rotation, depending on the position and track of the groove 216. These speed differences can also be expressed in differences in acceleration (positive or negative). Commonly, a negative acceleration is also termed deceleration.

Upon anti-clockwise rotation of the rotating member 204, an inserter device with a rotating member 204 similar to the one depicted in FIG. 6B, will show a higher acceleration and higher speed of insertion of the piston 241 within the first ˜90° rotation than an inserter device with a rotating member 204 similar to the one depicted in FIG. 6C. This is due to the steeper track of the groove 216 (FIG. 6B) compared to the track's slope seen in FIG. 6C. However, during the remaining ˜90° rotation, the insertion speed will be opposite. But with similar rotation speeds, similar height of the rotating member and similar lead, the total time needed for insertion will be similar, though the respective speeds of insertion, and also the respective accelerations and decelerations will be different. These are directly proportional the slope of the groove 216 of the rotating member 204.

FIG. 7 shows a semi-transparent view of the longitudinal moving means 241, comprising a piston 242, an insertion needle 243, one or more longitudinal guiding means 244, positioned symmetrically and diametrically towards each other, and transformation means 246. In the depicted embodiment, the transformation means 246 are provided in essentially the shape of a small cylinder or rod, protruding radially inwards from the inner wall of the piston 242, positioned close to the top of the piston 242, opposite the guiding means 244. The transformation means 246 are provided with a diameter, length and shape that allows said transformation means 246 to fit and remain in the grove 216 of the rotating member 204, and to transform the rotation of the rotating member 204 into a longitudinal movement of the longitudinal moving means 241.

The longitudinal guiding means 244 can be of essentially rectangular shape, and they fit into the groove of the guiding means 226, provided within the central cavity of the middle section 221 (see e.g. FIG. 3). In one embodiment, the longitudinal guiding means 244 are approximately of the same length as the height of the piston 242. In another embodiment, the length of the longitudinal guiding means 244 are longer than the length of the longitudinal movement. In a further embodiment, the length of the longitudinal guiding means 244 exceeds the height of the rotating member 204. In yet another embodiment, the length of the piston 242 is greater than the height of the rotating member 204.

FIG. 8 reveals an embodiment of an inserter device 200 according to the invention. For clarity, some sections or part of sections are either removed and/or shown as cross-sections. FIG. 8 gives an impression of the different dimensions and shapes of the major constituents of an inserter device according to the invention.

The activation means 261 are not visible in FIG. 8.

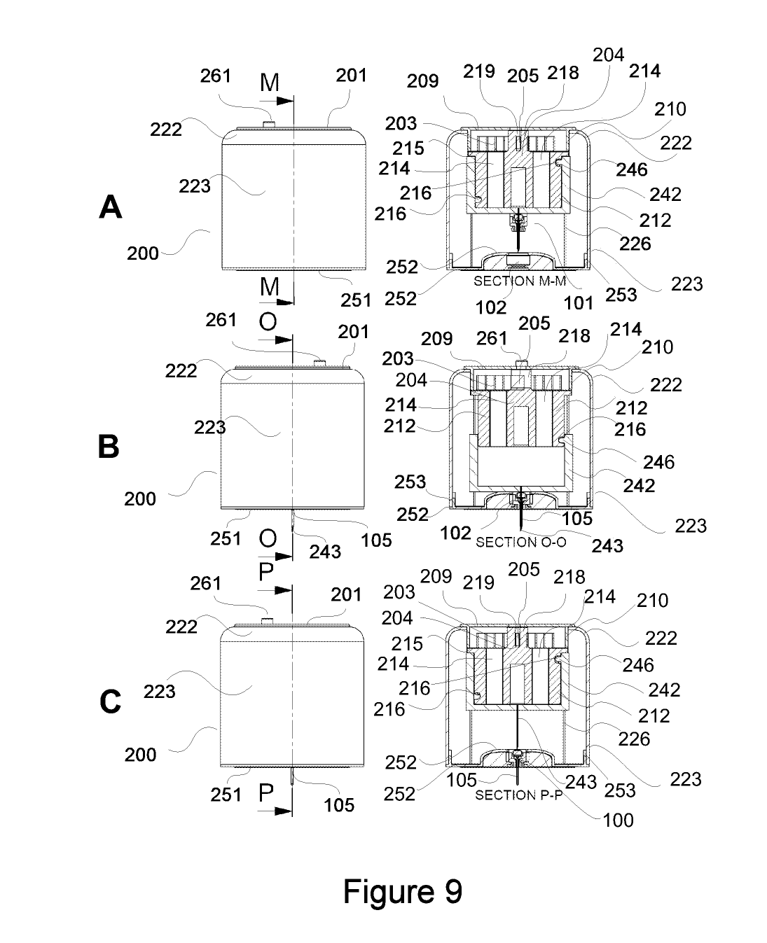

FIG. 9 presents 3 design drawings showing cross sections of an embodiment of an inserter device 200 according to the invention. The relative positions, proportions and interaction of the major constituents are illustrated. The inserter device has several features in common with the inserter devices 200 presented in the previous Figures (FIGS. 2-8). The respective views are indicated by letters and arrows. The numbering of the different constituents of the inserter device is indicated, and coherent with the sections above.

In FIG. 9A, the inserter device 200 is loaded/activated/energized and ready for insertion. This view reveals the shape of the longitudinal channels or cavities or openings 214, which extend across the rotating member 204. A central cavity becomes apparent, which is located towards the lower section of the rotating member 214. It is positioned in continuation of the shaft 218, and approximately as wide as the diameter of the shaft.

FIG. 9B, the inserter device 200 is presented after an approximate 180° rotation of the rotating member 204. The diameter of the spiral spring 203 has increased, and cannula holding part 101 has been brought in position within the body 102 of the medical device, and cannula 105 and the tip of the insertion needle 243 protrude the bottom plane and bottom part 252.

FIG. 9C shows the inserter device 200 after insertion of the medical device 100. The insertion needle 243 retracted, and the inserted device is ready for removing from the patient.

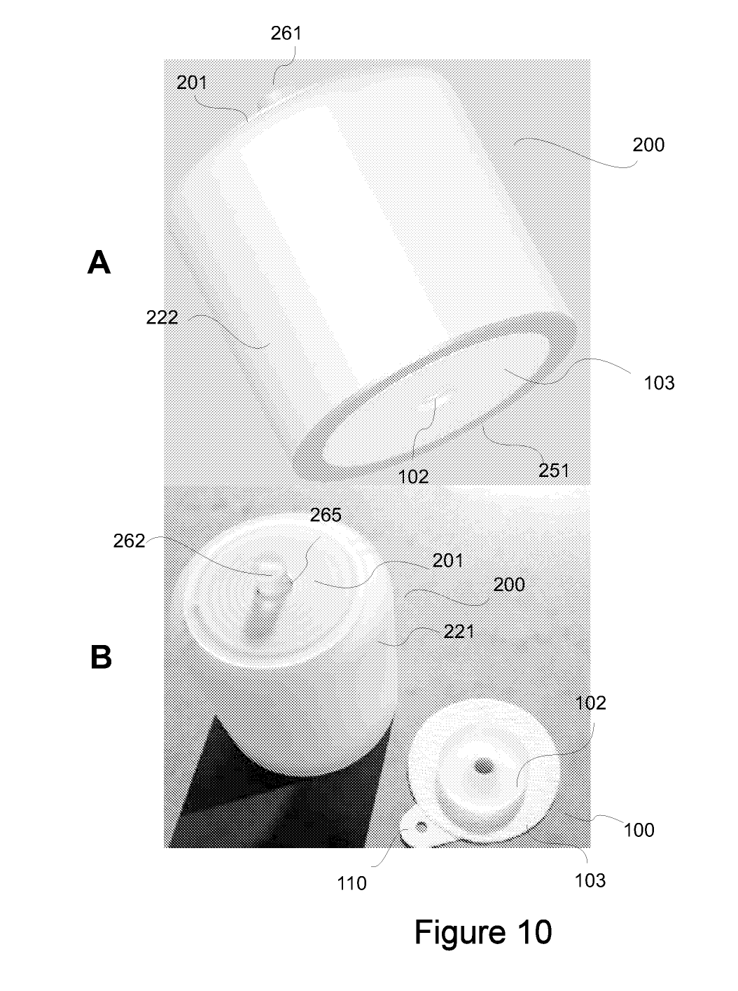

FIG. 10 shows further embodiments of an inserter device 200 according to the invention. FIG. 10A reveals the bottom section 251, where in this embodiment the release liner has been removed from the mounting pad 103, revealing the bottom surface of the release liner. Commonly, this surface will have a sufficient adhesive strength to provide sufficient adhesion to the skin of the patient in order to keep mounting pad, medical device and cannula in the desired position. When appropriate, said adhesive strength is also sufficient to allow the inserter device to be removed safely, while still keeping the medical device at the desired position.

FIG. 10 B provides a top view of an inserter device 200 with activation means 261. To the right, an embodiment of a medical device 100 is seen, comprising mounting pad, release liner with flap 112.

The diameter of the mounting pad 103 is of similar size or smaller than the outer diameter of the inserter device 200. In another embodiment of the invention, the diameter of the mounting pad 103 is larger than the outer diameter of the inserter device 200.

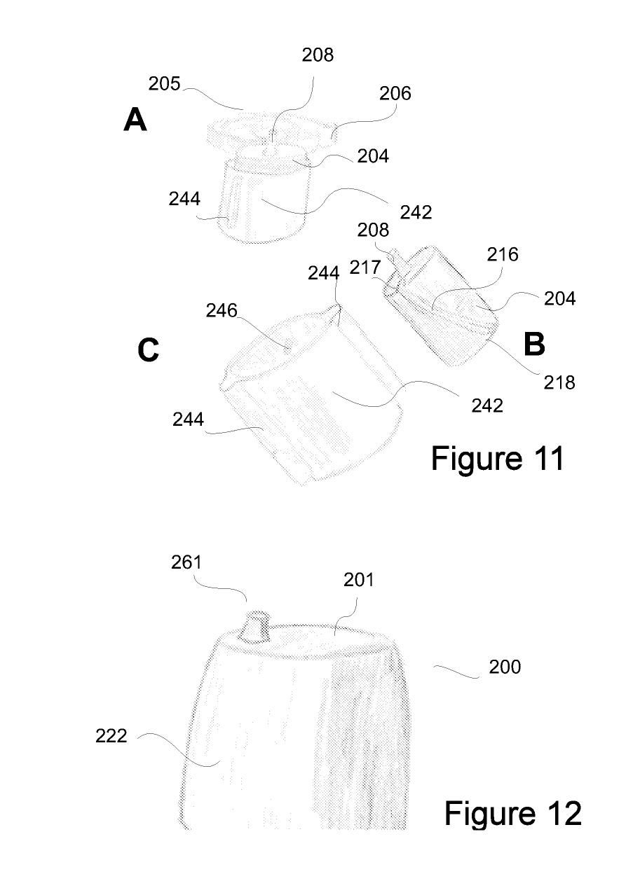

FIG. 11 shows alternative embodiments of an inserter device according to the invention. FIG. 11A shows an embodiment of rotating means 202, comprising a spiral spring 205 and a rotating member 204, and a piston 242. In this embodiment, the shaft 208 is longer than the width of the spiral spring 203. The longitudinal guiding means 244 are slightly rounded. In this embodiment, the longitudinal guiding means 244 shorter than the height of the piston 242. Note that the rotating member 204 does not comprise a top part 213 and no opening(s) 214.

FIG. 11B shows an embodiment of a rotating member 204 with a groove a shaft 208 and a groove 216. Also this embodiment of a rotating member does not comprise a top part 213, and has no opening 214. The track of the groove 216 is very step (high lead), going from the upper end 217 to the lower end 218 of the rotating member 204 within approximately 90°. An inserter device 200 according to the invention with a rotating member 204 will provide insertion and or retraction of a medical device to be inserted into a patient with less rotation than for example an inserter device 200 comprising a rotating member 204 as shown in FIG. 2, 3, 5, 6, 8 or 9. In these embodiments, a rotation of approximately 180° of a rotating member 204 is converted or transformed into a complete insertion movement in longitudinal direction of a longitudinally moving member 241.

However, in another embodiment of the invention, a groove 216 is provided with a track requiring more than 180° for insertion or retraction or both, such as 181° to 360°, or more than 1 rotation, turn or revolution, i.e. more than 360°, such as 361-540, or more than 1.5 rotations, or more than 2 rotations.

In a further embodiment, the degree of rotation required of the rotating member 204 for insertion and/or retraction is less than 180°, such as 10° to 170°, or 20° to 160°, or 30° to 160°, or 40° to 150°, or 50° to 140°, or 60° to 130°, or 60° to 120°, or 70° to 110°, or 80° to 100° or around 90°.

In yet a further embodiment, the degree of rotation required for insertion is essentially the same as the degree of rotation required for retraction.

In yet another embodiment, the degree of rotation required for insertion is different from the degree of rotation required for retraction, such as more than +/−5°, more than +/−10°, more than +/20, more than +/−45°, more than +/−90°, more than +/−135°, more than +/−180°, more than +/−270°, or more than +/−360°.

In still another embodiment, the groove 216 crosses itself, i.e. the track for the downward movement and the following upwards movement. This can be required when rotations of more than 360° are required for a complete insertion and retraction.

In an alternative embodiment, the longitudinal retraction movement is achieved by rotating the rotating member in the opposite direction.

FIG. 11C shows an embodiment of a piston 242 according to the invention. The longitudinal guiding means 244 possess a rounded cross section, and extend from the top of the guiding means almost to the bottom of the piston 242.

FIG. 12 shows an embodiment of an inserter device 200 according to the invention, and in this view, the middle section 221 can be seen, as well as activation means 261, comprising a button 262, provided of centre of the top section 201. It becomes apparent that the mechanical layout of the inserter device with the dominating middle section 221 opens up for a variety of design and feature possibilities.

Although depicted essentially flat in FIG. 12 and previous Figures, top section 201 can also be rounded, of symmetrical or asymmetrical shape. Thus, top section 201 may also comprise one or more protrusions or one or more notches or grooves.

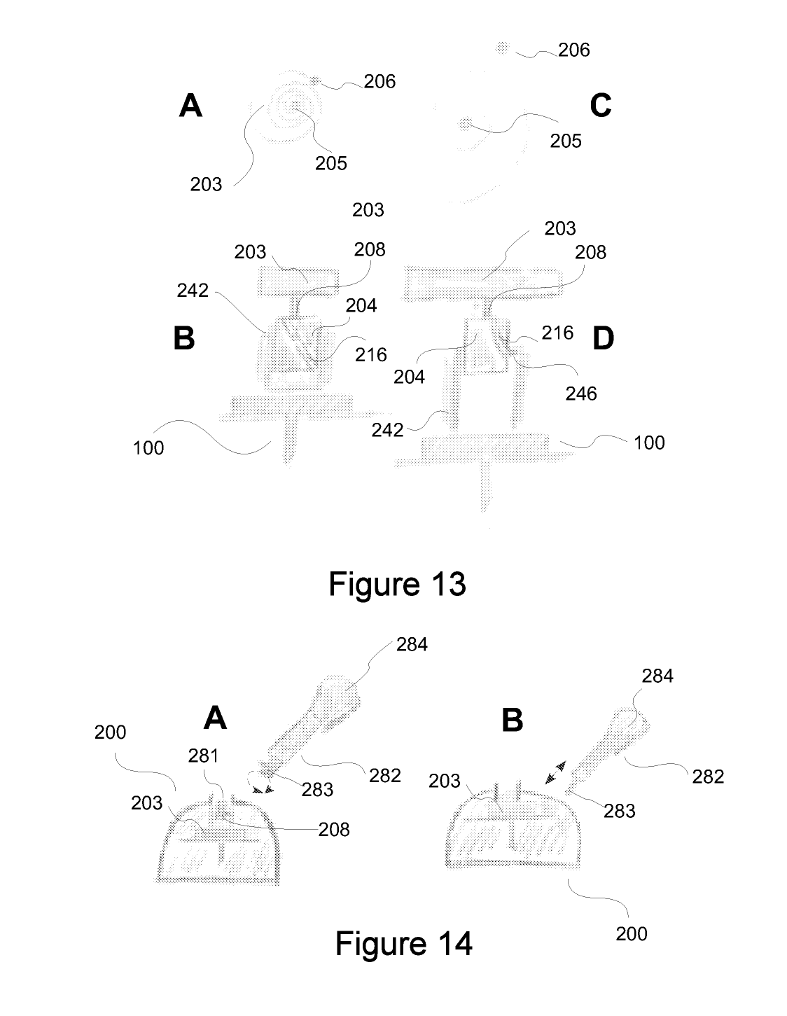

FIG. 13 A, B, C and D summarizes the first aspect of the invention, concerning an inserter device, where a rotational movement is transformed into a longitudinal insertion movement, wherein the axis of said rotational movement and the axis of said longitudinal insertion movement are essentially parallel and overlapping. The drawings are not to scale.

FIG. 13 A shows an embodiment of an activated, tense and compacted spiral spring 203, the spring 203 is fastened to the shaft 208 at the centre 205 and to the not shown housing 223 at the periphery 206. FIG. 13 B shows a partial cross-section of an inserter device according the invention where the energy providing means comprises a spiral spring 203, before, or at the onset of insertion. FIG. 13 C shows the same embodiment of a spiral spring 203 as shown in FIG. 13 A but in FIG. 13 C the spring 203 is in a relaxed, expanded state. FIG. 13 D shows a partial cross-section of the same embodiment of an inserter device as shown in FIG. 13 B, during, or at towards the end of insertion.

The diameter of the spiral spring 203 which is shown in FIG. 13 A, B, C and D increases during relaxation. In another embodiment of the invention, the diameter of the spiral spring decreases, when moving from the activated state to the relaxed state. The control of insertion speed is provided by the strength or amount of energy released by the energy providing means per time interval, together with technical features for controlling the amount of energy released per time interval. Further, the speed and changes of speed and direction can be controlled by the lean, i.e. slope of the groove 216 of the rotating member 204, which is directly transformed to a longitudinal movement of a longitudinally moving member 242.

In one embodiment of the invention, the energy providing means for providing energy for insertion of the medical device 100 comprises a clockwork. In a further embodiment, the clockwork comprises controlling means for providing a controlled release of energy. Release of energy can be constant or essentially constant. Alternatively, the release of energy can be varying during insertion of the medical device 100. Furthermore, if required, the energy provided for retraction an insertion needle 243 can be different from the energy for inserting the medical device 100.

FIG. 14 shows two alternative possibilities for activating an inserter device according to the present invention. FIG. 14 shows an inserter device 200, comprising energizing means. Said energizing means comprise interacting means 281, which interacting means 281 are connected to the shaft 208 of the inserter device, and a key 282 comprising a handle 284 and connecting means 283, said connecting means 283 can interact with the interacting means 281, in order to energize or activate an energy storage device, such as a spiral spring 203.

In FIG. 14 A shows an embodiment, where external, manual or automated input, such as rotation of the key 282 is transformed into a rotation which leads to activation or inactivation of the spiral spring 203. The rounded arrow indicates said rotation of the key 282. The automated input can also comprise one or more of electric, electronic and electromagnetic input. In one embodiment of the invention, an electric/electromagnetic motor provides activation of the spiral spring 203. In another embodiment of the invention, the external input comprises compressed gas, such as compressed air, CO₂, N₂ and the like.

In FIG. 14 B, the energy storage device (spiral spring 203) is activated by manual or automated input, said input being in a longitudinal up or down movement, or a combination of up and down movements, as indicated by the double arrow. According to one embodiment of the invention, external energy provides the required energy for insertion of a medical device 100, and optional retraction of an insertion needle 243. In another embodiment, external energy provides the energy required for bringing spiral spring 203 from a relaxed state to an activated state.

### Rotation Axis and Insertion Axis are Perpendicular

A second aspect of the invention relates to an inserter device, where a rotational movement is transformed into a longitudinal insertion movement, wherein the axis of said rotational movement and the axis of said longitudinal insertion movement are essentially perpendicular to each other.

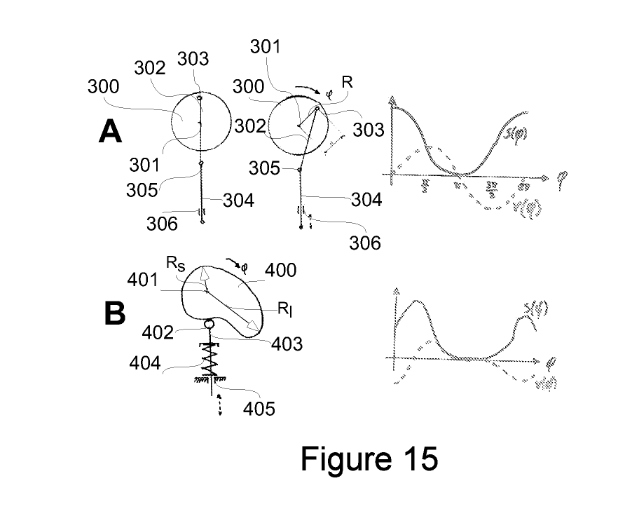

FIG. 15 illustrates two different principles of converting a rotational movement into a longitudinal movement. A common feature for both principles is that the direction of the longitudinal movement is not parallel to the rotation axis of the rotating member, in contrast to the first aspect of the invention. Commonly, the direction and axis of the longitudinal movement is perpendicular, or essentially perpendicular or orthogonal to the rotation axis of the rotating member.

FIG. 15A illustrates how the rotation of a rotating member, such as a disk or wheel can be converted into a longitudinal movement. Furthermore, the corresponding functions of translocation (S) and velocity (V) are shown schematically, each as a function of rotation (φ). It is seen that these functions are essentially sine or cosine functions, with a period of 2π. Using this principle for providing a controlled and defined longitudinal movement, speed and acceleration are defined by the above mentioned trigonometric functions (sin and cos) and are dependent on the speed of rotation.

The technical means for providing a longitudinal movement from a rotational movement comprise:
a rotating member 300 rotating around an axis 301—in this embodiment shown as a rotating disk, however virtually any other rotating body could do as well.
a first elongated member 302, such as a connecting rod
attachment means 303—connecting the first elongated member to said rotating member 300 and providing a pivoting movement of the first elongated member 302 as required.
a second elongated member 304 (piston)
a joint 305 between first and second elongated member, providing a pivoting movement between the first 302 and the second elongated member 304 as required, as well a longitudinal movement in the direction of the second elongated member 304.
longitudinal guiding means 306, which provide and govern the alignment of the second elongated member 304 in the direction of the desired longitudinal movement.

The length of insertion/longitudinal movement is essentially as long as 2× the distance from the centre of rotation to the point of attachment of first elongated member.

FIG. 15B illustrates a different principle for transforming a rotational movement of a rotating member into a defined linear movement of longitudinally moving member. In this embodiment, a rotating member, of irregular shape defines a resulting longitudinal movement. The corresponding graphs of translocation (S) and velocity (V) are shown schematically, each as a function of rotation (φ).

The technical means for providing a longitudinal movement from a rotational movement comprise:
a rotating member 400 rotating around an rotating axis 401—in this embodiment a rotating disk of irregular shape; alternatively, a circular disk, rotating off centre will also provide.
transformation means 402
an elongated member 403
longitudinal guiding means 405
elastic means 404, for example comprising a spiral spring.

The length of insertion/longitudinal movement is defined by the differences in radius, i.e. essentially as long as the difference between the rotating member's 400 longest radius (Rₗ) and shortest radius (Rₛ)

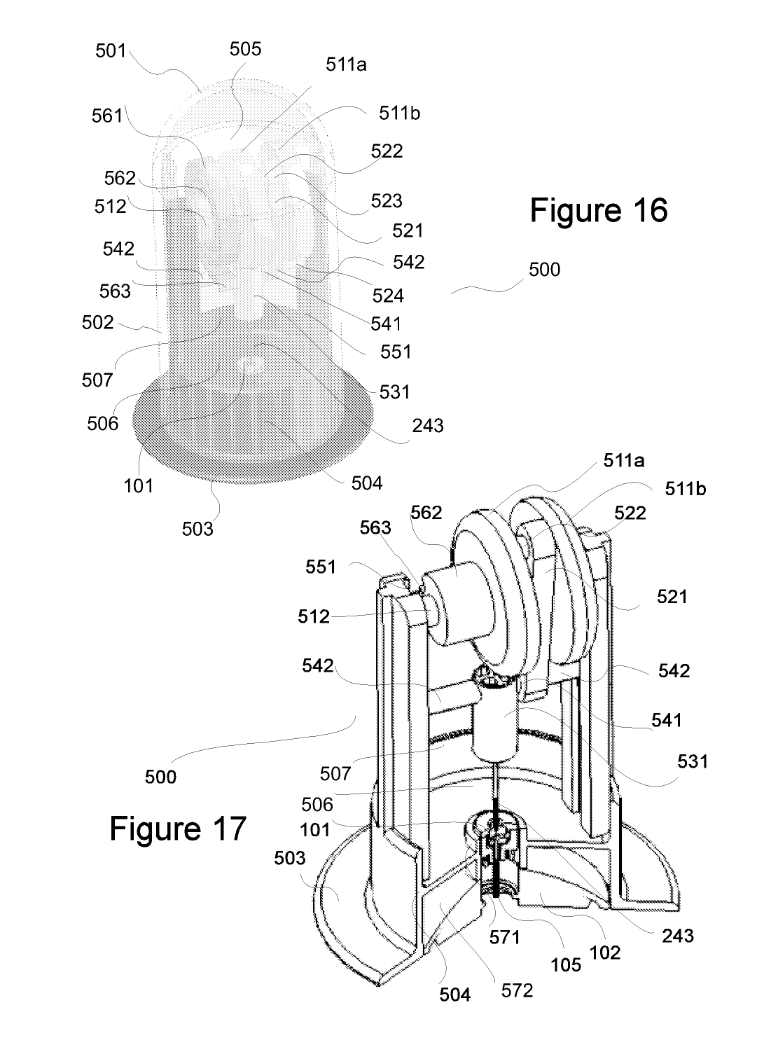

The following Figures (FIG. 16 to FIG. 19) illustrate embodiments of the current invention, based on the principle of transforming a rotational movement into a longitudinal movement, as previously presented in FIG. 15A.

FIG. 16 shows a semi-transparent view of an embodiment of an inserter device 500 with crank shaft according to the present invention. The inserter device' 500 comprises a top section 501, a middle section 502 and a body section 503, which govern the appearance of the inserter device. A central cavity 505 is provided within top section 501, middle section 502 and body section 503.

Top section 501 is rounded and of the shape of a half-sphere. Top section 501 and middle section 502 are connected, and can be provided in one piece or as separate pieces, which have to be joined. Top section 501 and middle section 502 have similar or matching wall thickness. Middle section 502 is of hollow-cylindrical shape. Commonly, although depicted semi-transparent, top section 501 and middle section 502 are not transparent.

Bottom section 503 is disk-shaped, and its diameter is larger than the outer diameter of the middle section 503. The bottom section comprises an inner extension 504. Middle section 502 overlaps the inner platform 504. The inner extension 504 comprises a platform 506, pair of guiding means 551, and a ring-shaped circular part 507.

The inner extension 504 of the bottom section 503 comprises an inner platform 506, which is essentially cylindrical; the outer diameter of said inner platform 504 is larger than the inner diameter of the middle section 502.

The guiding means 551 are positioned diametrically towards the outside of the inner extension 504, extending vertically upwards (i.e. orthogonally to the bottom plane of the inserter device 500) from the inner platform 506, thus inside middle section 502 and the central cavity 505.

At the centre of the inner platform 506, there is an opening, where the cannula holding part 101 is seen, with the cannula pointing downwards, in the direction and axis of insertion.

At the upper end of the guiding means 551, a crank shaft 512 is provided with bearing means (not shown), allowing for rotation of the crank shaft 512 parallel to the bottom plane of the inserter device, i.e. horizontally, and perpendicularly to the guiding means 551.

On crank shaft 512, within the two guiding means 551, a spiral spring 561 and two discs 511a and 511b are attached orthogonally and concentrically onto said crank shaft 512, so that crank shaft 512, spiral spring 561 and discs 511a and b share the same rotation axis. Discs 511a and b, as well as spiral spring 561 are of similar diameters. Disc 511a is mounted near the centre of the crank shaft 512, flanked by disc 511b and spiral spring 561. Only the inner part 562 of spiral spring 561 is attached to the crank shaft 512. Crank shaft 512 can comprise two sections, so that on the one section of the crank shaft 512 are mounted spiral spring 561 as well as disc 511a, while disc 511b is mounted on the other section of crank shaft 512.

Attachment means 522 are provided between the two discs 511a and b, said attachment means 522 connecting rod 521 with the discs 511a and b, and allowing for a pivoting movement of the connecting rod 521. The connecting rod 521 is attached off-centre of the discs 511a and b. The attachment means 522 comprise of a second shaft, mounted parallel to said crank shaft 512 via a through-going opening, near the top end 523 of the connecting rod, and the second shaft protruding said connecting rod on both sides of the trough-going opening.

Apart from providing a point of attachment of the connecting rod 521, the attachment means 522 provide a stable connection between discs 511a and b, so that a rotation of the crank shaft 512 is carried on from on disc 511 to the other, also in the absence of a section of the crank shaft 512. This provides the necessary space for the connection rod 521 to transform a rotation of discs 511a and b into a pivoting and up-and downwards movement, which would otherwise be partially restrained or interfere, if crank shaft 512 would be an ordinary by shaft, such as a rotating rod or cylinder.

Near the bottom end 524 of the connection rod 521, a flexible joint 541 is provided for connecting the bottom end 524 of connecting rod 521 with piston 531. In the depicted embodiment, the pivoting movement of the connecting rod 521 is transformed into a longitudinal movement of the piston via said flexible joint 541. Flexible joint 541 comprises a traversal bar 542, onto which piston 531 is attached in the centre of the traversal bar 542. The traversal bar 542 is parallel to the crank shaft 512, and remains parallel to shaft 542 while moving up and down as required, guided by the guiding means 551. Said guiding means 551 prevent the transversal bar from pivoting or twisting, for example around the axis of insertion.

The piston 531 is solidly attached to the transversal bar 542, and in this embodiment, the piston 531 is positioned in the centre of the transversal bar 542, aligned in direction of insertion and aligned with the centre axis of the insertion device 500.

At the bottom end of the piston 531, an introducer needle 243 is attached (tip pointing down), and said introducer needle being aligned with the centre axis of the insertion device 500. The tip of the introducer needle 243 is not visible, and in the depicted embodiment, it is introduced into the cannula holding part 101 of a medical device.

Another feature of the traversal bar 542 is that it provides attachment means 563 in the form of a fixing point of the outer end of the spiral spring 561, resulting in the outer end of the spiral spring 561 resting against the traversal bar 542.

FIG. 17 is a partial cross section of the embodiment of an inserter device 500 with crank shaft 512 as presented in FIG. 16, before or ready for insertion. For clarity, top section 501 and middle section 502 are removed. Furthermore, spiral spring 562 is not shown, whereupon spring attachment means 563 are seen, which in this embodiment comprise a longitudinal groove in crank shaft 512. The partial cross section through bottom section 503 and inner extension 504 reveal a bottom cavity 572, which provides room for the body 102 of a medical device to be inserted. The top of bottom cavity 572 is defined by the bottom surface of platform 506, the sides of the bottom cavity 572 is defined by the inner surface of the inner extension 504, the bottom of the bottom cavity 572 is defined by the body 102 of the medical device to be inserted, and the central section of the bottom cavity 572 is defined by the outer surface of cannula holding part guiding means 571. The function of said cannula holding part guiding means is to maintain the cannula holding part oriented in the direction of insertion before and during at least a part of its insertion.

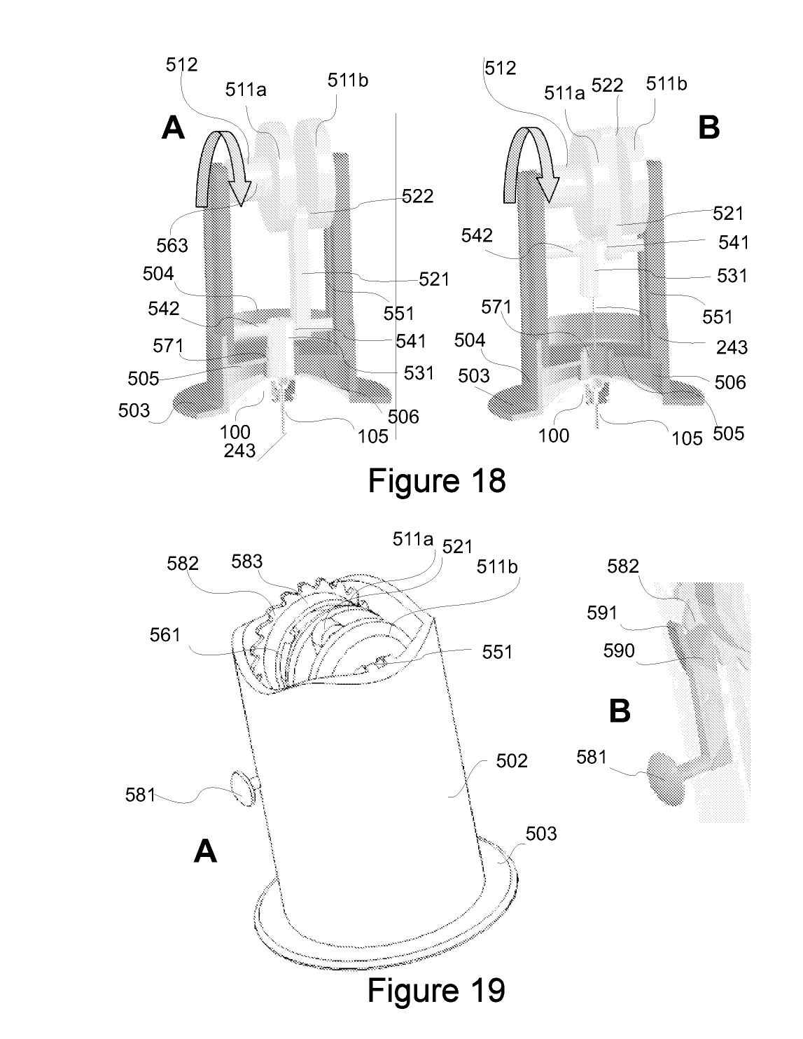

FIG. 18 show the embodiment of an inserter device 500 with crank shaft, as previously presented in FIGS. 16 and 17 in an inserted (A) and in a retracted (B) state. The numbering corresponds to the numbers and nomenclatures presented earlier, and the direction of rotation is indicated. In this embodiment, the direction of rotation for insertion and retraction are the same. In another embodiment, direction of rotation and insertion are different. This figure illustrates how the different moving parts interact in order to transform rotation into a translatory, longitudinal movement, resulting in the insertion of a penetrating member, followed by retraction of an inserter needle 243. The similarities between the depicted embodiment, and the general principle outlined an introduced in FIG. 15A become obvious in the following Table I:

TABLE I
Comparison between FIG. 15A and FIG. 18

| FIG. 15A | FIG. 18 |
|---|---|
| Rotating member 300 | Discs 511a and b, crank shaft 512 |
| Axis 301 | Rotation axis of crank shaft 512 |
| | and discs 511a and b |
| First elongated member 302 | Connecting rod 521 |
| Attachment means 303 | Attachment means 522 |
| Second elongated member 304 | Piston 531 |
| Joint 305 | Joint 541, transversal bar 542 |
| Longitudinal guiding means 306 | Guiding means 551, cannula |
| | holding part guiding means 571 |

FIG. 19 illustrates another embodiment of an insertion device according to the invention with crankshaft. In FIG. 19A the top section 501 and a part of middle section 502 are removed, revealing following features: A toothed wheel 582 is provided with a cover 583 surrounding the spiral spring 561. Furthermore, activation means 581 are seen. In this embodiment, the activation means are situated externally, and protrude middle section 502.

Activation means 581 comprise a button and a shaft. FIG. 19B shows a detailed view of an embodiment of the activation means 581. The activation means 581 comprise a button situated on a shaft protruding from a rocking leaver 590. In the depicted position of the activation means 583, the tip of the rocking leaver 590 fits into a groove between two neighbouring teeth of the toothed wheel 582. An integrated spring 591 is provided on said rocking leaver 590. The integrated spring is positioned between the inner wall of middle section 502 and the teeth of toothed wheel 582. In the depicted position in FIG. 19B, the toothed wheel 582 cannot turn. Upon application of a downwards force on said button in direction of said shaft, the rocking leaver pivotes, and as a result the tip of the rocking leaver 590 is no longer situated between the teeth of the teethed wheel 582. Thereby, the insertion device 500 is activated and insertion is initiated.

An inserter device according to the present application commonly comprise an opening at the bottom, which is sufficiently wide to allow a medical device 100 to leave the inserter device through said opening.

In another embodiment of the invention, the opening can be sealed with a detachable sealing foil, which may comprise a flap in order to facilitate the removal process before use of the inserter device. Such a detachable sealing foil is not necessarily a part of the mounting pad 103 of a medical device 100. The detachable sealing foil, or a mounting pad 103 with release liner can ensure an appropriate hygiene standard, by maintaining appropriate levels of disinfection or sterility. Furthermore, the sealing foil may act as an indicator for integrity of the inserter device and/or medical device 100, thereby improving safety standards, as use of potentially compromised and thus no longer sterile device can be avoided.

1. An inserter device for inserting a penetrating member into the subcutaneous and/or intramuscular area of a patient, said inserter device comprising:
a housing encompassing a penetrating member, the penetrating member comprising a soft cannula and an introducer needle, the introducer needle comprising part of the inserter device,
a rotating member, the rotating member comprising transformation means transforming the rotational movement into a longitudinal movement of the penetrating member in a direction of insertion and the transformation means comprises controlling means providing a controlled variation of a velocity of the penetrating member in the direction of insertion:
and driving means for rotating the rotating member around a rotating axis;
wherein continued rotation of the rotating member in the same direction of rotation or rotation of the rotating member in the opposite direction of rotation after insertion of the penetrating member provides that insertion of the penetrating member is followed by retraction of the introducer needle.

2. An inserter device according to claim 1, wherein the rotating member's rotation axis is parallel to the direction of insertion of the penetrating member.

3. An inserter device according to claim 2, wherein the rotating member's rotation axis is aligned with the direction of insertion of the penetrating member.

4. An inserter device according to claim 2, wherein the transformation means comprises a groove on a surface of a body part of the rotating member corresponding to a protruding part connected to the penetrating member.

5. An inserter device according to claim 2, wherein the controlling means comprises a slope of the groove as the groove extends in a direction which is not parallel to the direction of insertion.

6. An inserter device according to claim 4, claim 4, wherein the groove is continuous and a slope of the groove is defined in a system of coordinates having an ordinate axis parallel to the rotation axis of the rotation member.

7. An inserter device according to claim 6, wherein at least a part of the groove has a negative slope or a constant negative slope in a whole length of the groove when providing a movement where the longitudinal moving member is moving towards the skin of the patient.

8. An inserter device according to claim 7, wherein the negative slope of the groove is decreasing as the longitudinal moving member moves toward the skin of the patient.

9. An inserter device according to claim 4, wherein the groove is continuous and at least a part of the groove has a positive slope or the groove has a constant positive slope in a whole length of the groove in a system of coordinates having an ordinate axis parallel to the rotation axis of the rotation member, when providing a movement where the longitudinal moving member is moving away from the skin of the patient.

10. An inserter device according to claim 9, wherein the positive slope of the groove is decreasing as the longitudinal moving member moves away from the skin of the patient.

11. An inserter device according to claim 4, wherein the body part of the rotating member is cylindrical and the groove is formed in the outer surface of the body part, the transformation means are formed as an inward protruding part on an inner surface of the longitudinal moving member.

12. An inserter device according to any of the claim 1, wherein the rotating member's rotation axis is not parallel to the direction of insertion of the penetrating member.

13. An inserter device according to claim 12, wherein the rotating member's rotation axis is orthogonal to the direction of insertion of the penetrating member.

14. An inserter device according to claim 12, wherein the transformation means comprises a shaft comprising one or more discs protruding in relation to the shaft and a rigid bar transforming the rotation into a longitudinal movement of the penetrating member, the controlling means comprises a combination of 1) the distance between the rotation axis of the rotating member and the fastening point of the rigid bar to a disc, 2) the angle at which the rotation starts in relation to the insertion direction, and 3) the driving means.

15. An inserter device according to claim 14, wherein the shaft is a crank shaft provided with two discs that are attached orthogonally and concentrically onto said crank shaft, so that crank shaft and discs share the same rotation axis.

16. An inserter device according to claim 1, wherein the controlled variation of velocity is provided by rotating the rotating member more than 90°,

17. An inserter device according to claim 16, wherein the transformation means transform a rotation of the rotating member more than approximately 90° into a longitudinal movement, said longitudinal movement providing an insertion of the penetrating member.

18. An inserter device according to claim 17, wherein the transformation means transforms the rotation of the rotating member more than about 180°.
